(19) United States
(12) Patent Application Publication
(10) Pub. No.: US 2018/0289272 A1
(43) Pub. Date: Oct. 11, 2018

(54) System for Processing Analog Neurological Monitoring Signals Including Digital Transmission

(71) Applicant: Rhythmlink International, LLC, Columbia, SC (US)

(72) Inventor: Daniel McCoy, Murrells Inlet, SC (US)

(73) Assignee: Rhythmlink International, LLC

(21) Appl. No.: 15/950,751

(22) Filed: Apr. 11, 2018

Related U.S. Application Data
(60) Provisional application No. 62/483,995, filed on Apr. 11, 2017.

(51) Int. Cl. A61B 5/04; H03M 1/12; G16H 10/60

(52) U.S. Cl. CPC A61B 5/04001; H04W 72/14; G16H 10/60; H03M 1/12

(57) ABSTRACT

A system includes a monitoring electrode for neurological monitoring and operable to generate an analog electrical signal from brain activity of a patient; an analog-to-digital converter in electrical connection with the monitoring electrode; a transmitter in electrical connection with the analog-to-digital converter for transmitting a digital electrical signal corresponding to the analog signal; a receiver in electrical communication with the transmitter and operable to receive the transmission of the digital electrical signal; and a digital-to-analog converter in electrical connection with the receiver for converting the digital electrical signal received by the receiver back to an analog electrical signal for processing by an analog amplifier.

### TECHNOLOGICAL FIELD

This disclosure relates to the field of neurological monitoring and, more particularly, to the collection, transmission, and processing of analog signals from neurological monitoring electrodes.

### BACKGROUND

Electrode monitoring, which is widely used in providing hospital treatment, involves the attachment of electrodes to the body of a patient, including head, face, extremities, and chest. In neurological monitoring, the electrodes may be attached to specific locations of the patient's scalp so that the results of monitoring can be evaluated, so that subsequent results from the same patient can be compared, and so that the results from monitoring different patients can also be compared to each other.

The electrodes respond to electrical signals in the brain by generating corresponding electrical signals that are transmitted to an amplifier, typically through an electrically conductive wire. The amplifier boosts and displays the electrical signals received.

The number of electrodes used with a patient may vary somewhat depending on the purpose of the monitoring. There are conventions for the numbers and locations of the electrodes to be used, such as the “10-20” standard of the American Electroencephalographic Society. In addition, there may be custom configurations used by specific individuals or organizations or used in evaluating a patient for particular health risks, such as stroke and traumatic brain injury.

To help manage the process of attaching the electrodes to locations on the scalp of the patient, templates or harnesses may be used to designate the locations for the electrodes; harnesses may include electrode in pre-selected relative positions.

When many electrodes are used, the large number of wires that are required to connect the electrodes to an amplifier pose several problems. First, connecting and disconnecting wires to electrodes and amplifiers is time-consuming. Second, the wires may interfere with other medical procedures such as magnetic resonance imaging and computerized tomographic scans. The wires and electrodes may not be compatible with these procedures.

In order to avoid wires, many manufacturers of neurological monitoring equipment use proprietary digital processing circuitry that receives the analog signals from electrodes and converts them to digital signals for transmission to an amplifier.

There are wireless monitoring systems, such as that shown in FIG. 1 and in US Publication 2015/0257674, filed by Jordan Neuroscience, in which the template or harness 10 worn by a patient 14 has a converter 18 attached to it for receiving analog signals from electrical conductors 20 built into the straps 22 of a harness 10 and converting them using converter 18 from analog signals to digital signals for wireless transmission to a customized, digital amplifier 26 that is designed to receive and process wireless digital signals for digital processing and display. Legacy amplifiers cannot process digital signal.

A system that helps to solve the problems of wires in neurological monitoring and is compatible with standard analog input neurological monitoring equipment would be useful.

### SUMMARY

The disclosure teaches the use of an analog-to-digital (ND) converter and a digital-to-analog (D/A) converter in the process of the digital transmission and reception of neurological signals in an otherwise analog system. According to this system, a neurological electrode generates an analog signal from the signals sensed near the electrode on the patient's head. That analog signal is conducted via a short wire directly to an analog-to-digital converter for conversion from an analog signal to a digital signal. The digital signal is then transmitted wirelessly to a stand-alone receiver and digital-to-analog converter where it is received as a digital signal and converted back to an analog signal. Thereafter, it is fed to an analog amplifier for display in the same manner as the amplifier would any analog signal received directly from the same electrodes.

The individual wires from the electrodes in use may be collected at the headset and attached to a short, ribbon cable leading directly to the analog-to-digital converter and thence to a digital transmitter. The stand-alone digital receiver near the analog amplifier receives the wireless transmission of digital signals from that transmitter and conducts them in analog form to the analog amplifier for processing and display. These re-converted analog signals from each electrode may be connected by lead wires from the output of the digital-to-analog stand-alone receiver directly to the correct input jack of a legacy amplifier.

In another aspect of the disclosure, the electrode itself may incorporate or be connected immediately to an analog-to-digital converter and a transmitter so that electrodes transmit wirelessly their digital signals to the remote receiver which then converts received digital signals back to analog signals and forwards them via wire to the analog amplifier.

An aspect of the disclosure is a device that includes a monitoring electrode that is attached to the patient and operable to generate an analog electrical signal from electrical currents generated in the body part to which it is attached; an analog-to-digital converter in electrical connection with the monitoring electrode; a transmitter in electrical connection with the analog-to-digital converter for transmitting the digital electrical signal; a receiver in electrical communication with the transmitter and operable to receive the transmission of the digital electrical signal; and a digital-to-analog converter in electrical connection with the receiver for converting the digital electrical signal received by the receiver back to an analog electrical signal.

Another aspect of the disclosure is a first electrical conductor between the monitoring electrode and the analog-to-digital converter.

Another aspect of the disclosure is that the analog-to-digital converter may be carried by a monitoring electrode or, alternatively, the monitoring electrode may be carried by the analog-to-digital converter.

An aspect of the disclosure is that the transmitter may be carried by the analog-to-digital converter or, alternatively, the analog-to-digital converter may be carried by the transmitter.

Another aspect of the disclosure is that the device may include a template operable to receive the monitoring electrode which template may also carry the analog-to-digital converter and the transmitter.

An aspect of the disclosure is that the receiver may carry the digital-to-analog converter or, alternatively, the digital-to-analog converter may carry the receiver.

An aspect of the disclosure is that the digital signal may be transmitted via radio modem technology such that certified by Bluetooth Sig, Inc.

These and other features and their advantages will be appreciated by those familiar with neurological monitoring and the equipment used therewith.

### BRIEF DESCRIPTION OF THE DRAWINGS

In the figures,

FIG. 1 is a view of a prior art device for neurological monitoring;

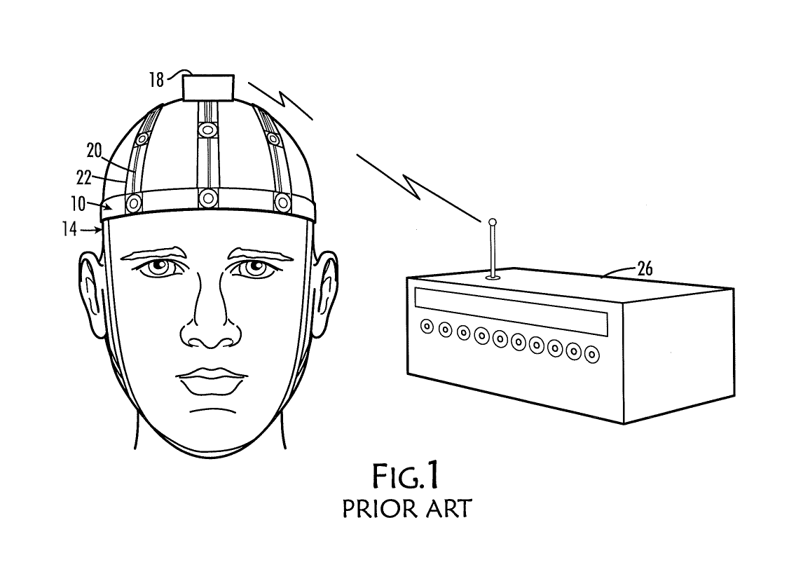

FIG. 2 shows the present device for neurological monitoring according to an aspect of the disclosure; and

FIG. 3 is a view of an alternative device for neurological monitoring, according to an aspect of the disclosure.

### DETAILED DESCRIPTION

The present system is for use with existing neurological analog amplifiers and neurological monitoring electrodes. Monitoring electrodes are used to sense small electrical currents in the part of a patient's body, such as in the brain. Neurological electrodes respond to the magnetic fields developed by the electrical currents in the brain by developing corresponding electrical currents that replicate the electrical signals of the brain. The electrical currents of the brain and elsewhere in the body and the electrical currents developed in response by the electrodes are analog currents. If these small analog currents from the neurological electrodes are transmitted wirelessly and as digital signals rather than analog signals, they will be less affected by destructive interference from electrical devices in the vicinity of the transmission.

The process of converting analog signals to digital is well known, as is the reverse process: converting digital to analog.

After analog electrical signals are converted to digital electrical signals, they can be transmitted by a transmitter to a remote receiver. The receiver receives these digital signals and converts them back to analog once again using a digital-to-analog converter so that they can be processed and displayed by an analog amplifier, such as a legacy amplifier. A legacy amplifier is an existing neurological amplifier that receives and processes analog signals. The term “processing” may include amplification, signal conditioning, and display.

Because there may be separate signals from each of, say, 20 or more electrodes, the transmission of each of the signals needs to be managed so that the signals can be transmitted and displayed properly. The integrity of each signal transmission, which means knowing from which electrode it came and that all of the data came from that signal, is part of that management process. Knowing the time the data was accumulated so that it can be synchronized with other date accumulated at the same time is important for display when the signals from two or more electrodes are to be compared.

Maintaining control and separation of the signals generated from the electrodes may be managed in various ways. For example, a signal from a specific electrode may be transmitted digitally at its own frequency and received by a receiver tuned that frequency. Alternatively, the digital signals can be multiplexed (by time or frequency division multiplexing) so that all twenty may be sent over the same conductor, with each electrode's signal representing one channel among the other nineteen channels, each signal digitized, and the data of all the signals added to a queue to be sent almost instantly. The signal from each channel may be coded with its own unique identifier. The transmitter may multiplex the data by frequency, with each channel being transmitted at its own frequency or by sampling each one of the analog signals in rapid succession and repeatedly to form a single transmission that contains information about all of signals. The digital-to-analog receiver may then receive and process the signal received, sorting the transmitted data on receipt into separate channels and converting the digital signals back to analog signals for display.

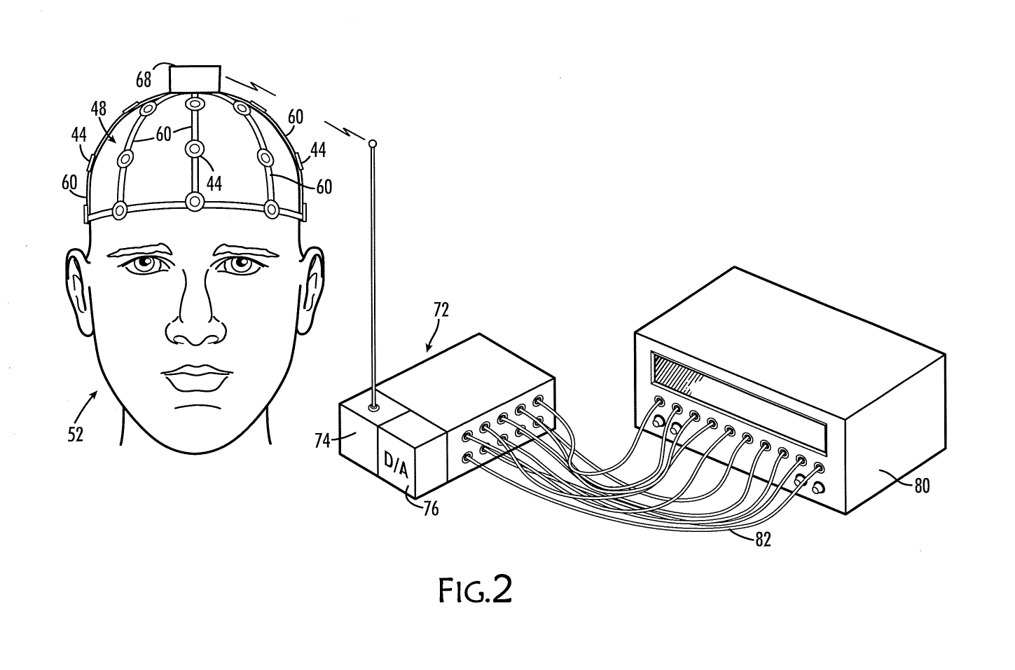

FIG. 2 shows the present system in which electrodes 44 are positioned on the scalp 48 of a patient 52 using a template 56 (or a harness) to facilitate the correct and complete positioning of the electrodes 44. Electrical conductors 60, such as insulated wires, may be used to place electrodes 44 in electrical connection with a nearby converter/transmitter 68 (identified in FIG. 2 as “A/D”) that convers analog signals to digital signals.

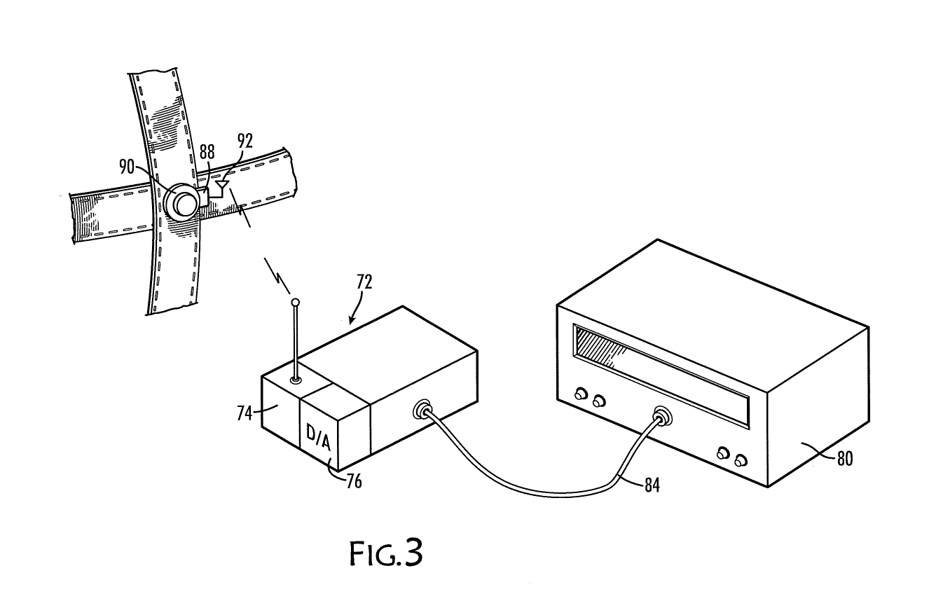

The digital signal is transmitted wirelessly by converter/transmitter 68 to a breakout box 72 that contains a receiver 74 and a digital-to-analog (D/A) converter 76. The signals received by receiver 74 of breakout box 72 are conducted to an amplifier/processor 80 in a manner the amplifier/processor is equipped to receive them. For example, in FIG. 2, amplifier/processor 80 receives the signals for each electrode from breakout box 72 via an individual wire 82. In FIG. 3, amplifier/processor 80 is equipped to receive the signals from each electrode through a single multi-pin cable 84.

Analog-to-digital converters are well-known electrical circuit element that convert electrical analog signals, which vary in amplitude, to digital signals that represent a series of instantaneous amplitudes of an amplitude-varying analog signal by a binary digital code. Similarly, a digital-to-analog converter, also a well-known electrical circuit element, converts a binary digital code to a magnitude that represents the amplitude of an analog signal.

As illustrated in FIG. 3, an analog-to-digital (A/D) converter 88 may be included as part of electrode 90 and also a transmitter 92 to eliminate the need for wires to run from each electrode 90 to an analog-to-digital (A/D) converter such as converter/transmitter 68 shown in FIG. 2. Transmitter 92 may be a miniature transmitter. A/D converter 88 generates a digital signal that is transmitted wirelessly via transmitter 92 to receiver 74 in breakout box 72. “BLUETOOTH” is a proprietary wireless technology standard for data exchange over short distances using ultra-high frequency band radio waves from 2.4 to 2.486 gigahertz, which may be useful for such a transmission. Alternatively, a small “dongle” can be part of or attached to electrode 90 that includes both ND converter 88 and transmitter 92.

Other connections between breakout box 72 and amplifier/processor 80 can be used, such as a ribbon cable.

The transmission of the digital signal may be made in any convenient manner, including a single transmission of multiplexed signals, or parallel transmissions at different frequencies, and other ways well-known to those of ordinary skill in the art. Also, each electrode may provide a unique signal to self-identify.

At breakout box 72, as shown in FIG. 2, which acts an interface, the wireless transmissions received, are associated with electrodes. The wireless transmissions are received as digital signals are then converted to analog signals and connect using individual wires 82 directly to the appropriate channels of amplifier/processor 80 for neurological monitoring.

Those skilled in neurological monitoring will appreciate that many substitutions and modification may be made to the foregoing description of aspects of the disclosure without departing from the spirit of the description.

What is claimed is:

1. A device, comprising:
a monitoring electrode attachable to a patient, said monitoring electrode operable to generate a first analog electrical signal from brain activity of said patient;
an analog-to-digital converter in electrical connection with said monitoring electrode, said analog-to-digital converter generating a digital electrical signal equivalent to said first analog electrical signal;
a transmitter in electrical connection with said analog-to-digital converter, said transmitter receiving and transmitting said digital electrical signal;
a receiver in electrical communication with said transmitter and operable to receive said digital electrical signal; and
a digital-to-analog converter in electrical connection with said receiver, said digital-to-analog converter converting said digital electrical signal to a second analog electrical signal equivalent to said first analog signal.

2. The device of claim 1, further comprising a first electrical conductor between said monitoring electrode and said analog-to-digital converter.

3. The device of claim 1, wherein said analog-to-digital converter is carried by said monitoring electrode.

4. The device of claim 1, wherein said transmitter is carried by said analog-to-digital converter.

5. The device of claim 1, wherein said analog-to-digital converter is carried by said transmitter.

6. The device of claim 1, further comprising a template carrying said monitoring electrode, and wherein said monitoring electrode carries said analog-to-digital converter and said template carries said transmitter.

7. The device of claim 1, wherein said receiver carries said digital-to-analog converter.

8. The device of claim 1, wherein said transmitter transmits said digital electric signal in an ultra-high frequency band at a frequency between 2.4 and 2.485 GHz.
